(19) United States
(12) Patent Application Publication
(10) Pub. No.: US 2011/0144749 A1
(43) Pub. Date: Jun. 16, 2011

(54) Cochlea Implant System in ITE (in the ear) Type Using Infrared Data Communication

(75) Inventors: Sung June Kim, I, Seoul (KR); Seung Ha Oh, Seoul (KR); Soon Kwan An, Seoul (KR); Hong Joo Lee, Gyeonggi-do (KR); Se-ik Park, Seoul (KR)

(73) Assignee: NUROBIOSYS, Gyeonggi-do, KR (KR)

(21) Appl. No.: 12/937,349

(22) PCT Filed: Aug. 25, 2008
(86) PCT No.: PCT/KR08/04951

§ 371 Date: Dec. 1, 2010

(30) Foreign Application Priority Data
Apr. 11, 2008 (KR) 1020080033760

(51) Int. Cl. A61F 2/18; A61N 1/00

(52) U.S. Cl. 623/10; 607/57

(57) ABSTRACT

There are provided a cochlea implant system including an in the ear (ITE) speech processor and an implanted part. In the cochlea implant system, the speech processor and the implanted part transfer and receive a signal by infrared data communication. The implanted part includes a receiving unit for receiving an infrared signal from the speech processor to demodulate the infrared signal, a stimulation circuit unit for converting the demodulated signal into a stimulation signal, an electrode array inserted into the cochlea to stimulate the auditory neurons by the stimulation signal, and a coil for receiving power from the outside through the RF power transmission. The implanted part can receive power from the outside through the coil to be charged.

### TECHNICAL FIELD

The present invention relates to a cochlea implant system including a speech processor and an implanted part. the speech processor and the implanted part communicate via infrared data communication. The implanted part receives its power from the outside through radio-frequency (RF) signal and/or recharges an implantable battery included in the ICS. For this RF power transmission, the speech processor and the implanted part are inductively linked by RF coils.

### BACKGROUND ART

A cochlear implant system is an artificial device for providing auditory sensation by electro-stimulation of remaining auditory neurons of a patient, who is severe-to-profoundly hearing impaired with sensori-neural origins

In general, a cochlea implant comprises a speech processor and an implanted part.

The speech processor provided outside the body processes the acoustic sound (a voice signal or an acoustic signal) received by a microphone and then, transfers the processed signal to the implanted part for the stimulation.

When the signal of the speech processor is received by the implanted part implanted into the body, a stimulation circuit unit converts the received signal into a proper stimulation signal including such information as electrode channel, stimulation mode, magnitude of stimulation, and period of stimulation to be suitable for stimulating the auditory neurons. The stimulation signal is delivered to the auditory neurons via an electrode array inserted into the patient's cochlea. The electrical stimulation is transmitted to the auditory cortex of a brain so that the hearing impaired can hear sounds.

### DISCLOSURE

### Technical Problem

The present invention relates to a cochlea implant system including a speech processor and an implanted part that transfer and receive a signal by infrared data communication. The implanted part includes a receiver for receiving an infrared signal from the speech processor to demodulate the infrared signal, a stimulation circuit unit for converting the demodulated signal into a stimulation signal, an electrode array inserted into the cochlea to stimulate the auditory neurons by the stimulation signal, and a coil for receiving power from the outside through radio frequency (RF) power transmission. The implanted part receives power from the outside through the coil to charge an internal battery.

The present invention has been made in view of the above problems, and it is an object of the present invention to provide a cochlea implant system using infrared data communication capable of being charged through the RF power transmission.

### Technical Solution

In accordance with an aspect of the present invention, the above and other objects can be accomplished by the provision of a cochlea implant system including a speech processor inserted into the external auditory canal to convert a voice signal or an acoustic signal into an electric signal, to process the electric signal, and to transfer the processed electric signal to an inside of a body, and an implanted part implanted into the mastoid cavity to receive power from an outside through radio frequency (RF) power transmission, to receive the signal from the speech processor, and to stimulate the auditory neurons in the cochlea implant system. Here, the speech processor and the implanted part transfer and receive the signal by infrared data communication.

Besides the infrared transmitter for delivering processed signal to the implanted part for stimulation, the speech processor can further include an infrared receiving unit in order to receive information on the status of an electrode array in the body for controlling a proper stimulation range when the cochlea implant system is applied, information on the auditory neural response of a patient in response to a test stimulus, and information on the operation status of the implanted part from the implanted part.

The implanted part includes a receiving unit for receiving an infrared signal from the speech processor to demodulate the infrared signal, a stimulation circuit unit for converting the demodulated signal into a stimulation signal, an electrode array inserted into the cochlea to stimulate the auditory neurons, a coil for receiving power from the outside through the RF power transmission, and a battery. The implanted part can operate directly by receiving power from the outside through the coil or can charge the battery included in the implanted part using the power received from the outside through the coil. In addition, the implanted part can further include an infrared transmitting unit and can transmit information on the status of an electrode array in the body for controlling a proper stimulation range when the cochlea implant system is applied, information on the auditory neural response of a patient in response to a test stimulus, and information on the operation status of the implanted part to the speech processor through the infrared transmitting unit in accordance with the command signal transmitted by the speech processor.

In addition, there is provided an implanted part implanted into the mastoid cavity to receive a signal from a speech processor and to stimulate the auditory neurons in a cochlea implant system. The implanted part includes a receiving unit for receiving an infrared signal from the speech processor through the skin of the external auditory canal to demodulate the infrared signal, a stimulation circuit unit for converting the demodulated signal into a stimulation signal, an electrode array inserted into the cochlea to stimulate the auditory neurons by the stimulation signal, a coil for receiving power from the outside through radio frequency (RF) power transmission, and a battery. In addition, the implanted part according to the present invention can further include a test circuit for extracting information on an electrode array status and a system operation status, a measuring circuit unit for measuring an auditory neural response induced by a test stimulus, and an infrared transmitting unit for transmitting information such as electrode array status, system operation status and auditory neural response to the speech processor.

In addition, there is provided a method of stimulating the auditory neurons including a speech processor converting a voice signal or an acoustic signal into an electric signal, modulating the converted electric signal into an infrared signal, transmitting the infrared signal to an implanted part implanted into the inside of the body through the skin of the external auditory canal, an implanted part receiving the transferred infrared signal, demodulating the received infrared signal, converting the demodulated signal into a waveform suitable for stimulating the auditory neurons, stimulating the auditory neurons by the converted signal, and receiving power through RF power transmission to charge an internal battery. In addition, the method can further include the implanted part measuring a stimulus waveform induced in an electrode array and a system internal operation signal and modulating the measured stimulus waveform and system internal operation signal, recording auditory neural response induced by an electric stimulus and modulating the recorded auditory neural response, and transmitting the modulated signal as the infrared signal to the outside.

### Advantageous Effects

In the cochlea implant system according to the present invention, the implanted part communicates with the speech processor through the infrared data communication and can receive power from the outside through the coil to be charged.

In particular, according to the present invention, the speech processor is inserted into the external auditory canal and the package of the implanted part is implanted into the mastoid cavity close to the posterior wall of the external auditory canal in order to perform remote infrared data communication with the speech processor inserted into the external auditory cavity.

On the other hand, the implanted part includes the coil so that the implanted part can be charged by the RF power transmission. The coil is implanted under the scalp above the temporal bone mastoid outside the external auditory canal.

Therefore, it is possible to minimize the size of the package to be implanted into the mastoid cavity of the body.

### DESCRIPTION OF DRAWINGS

The above and other objects, features and other advantages of the present invention will be more clearly understood from the following detailed description taken in conjunction with the accompanying drawings, in which:

FIG. 1 is a view illustrating that a cochlea implant system according to an embodiment of the present invention;

FIG. 2 is a block diagram of the cochlea implant system according to an embodiment of the present invention;

FIG. 3 is a view illustrating that the auditory neurons are stimulated by the cochlea implant system according to the present invention;

FIG. 4 is a view illustrating that a cochlea implant system according to another embodiment of the present invention;

FIG. 5 is a block diagram of the cochlea implant system according to another embodiment of the present invention;

FIG. 6 is a view illustrating processes of charging the implanted part of the cochlea implant system according to the present invention;

FIG. 7 is a view illustrating processes of installing a program in the implanted part of the cochlea implant system according to the present invention; and

FIG. 8 is a block diagram of a cochlea implant system capable of performing interactive communications between an implanted part and a speech processor according to still another embodiment of the present invention.

### BEST MODE

Hereinafter, embodiments of a cochlea implant system according to the present invention will be described in detail with reference to the accompanying drawings. However, the present invention is not limited to the following embodiments.

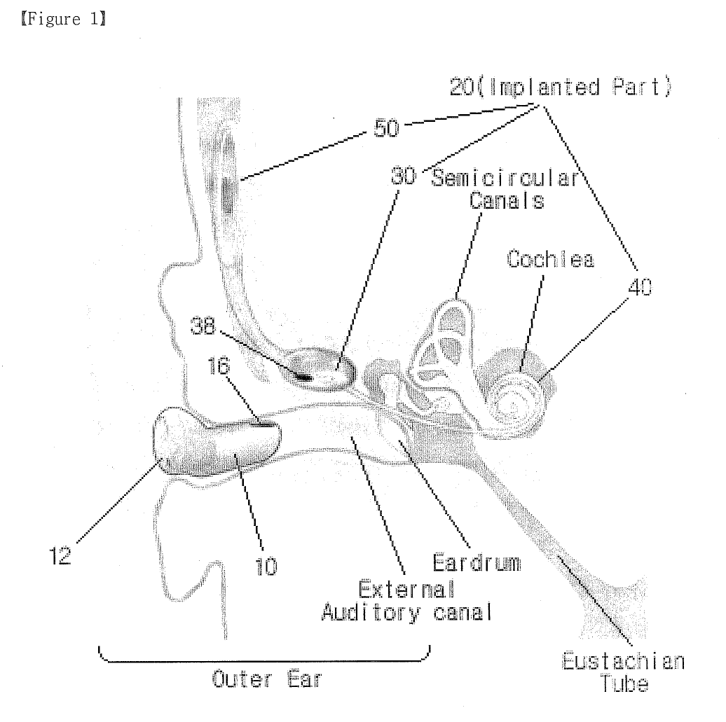

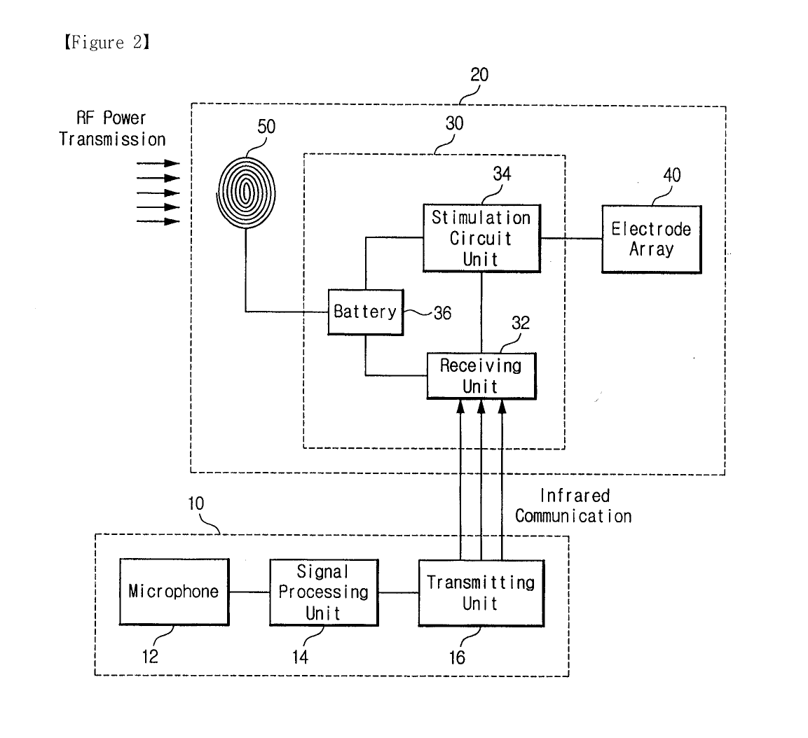

FIG. 1 is a view illustrating that a cochlea implant system according to an embodiment of the present invention. FIG. 2 is a block diagram of the cochlea implant system according to an embodiment of the present invention.

Referring to FIGS. 1 and 2, the cochlea implant system according to the present invention includes a speech processor 10 for converting a voice signal or an acoustic signal into an electric signal, for processing the electric signal, and for transmitting the processed electric signal to the inside of a body and an implanted part 20 for receiving the signal from the speech processor 10 and for stimulating the auditory neurons in the cochlea implant system.

The speech processor 10 and the implanted part 20 can transfer and receive the signal interactively by infrared data communication.

The speech processor 10 includes a microphone 12 for converting the voice signal or the acoustic signal into the electric signal and a transmitting unit 16 for modulating the electric signal, for converting the modulated electric signal into an infrared signal, and for transmitting the converted infrared signal to the inside of the body. The speech processor 10 can further include a signal processing unit 14 for digital signal processing the electric signal of the microphone and a battery (not shown) for driving the speech processor 10.

The speech processor 10 is manufactured to have a proper size so that the speech processor 10 can be inserted into the external auditory canal of a patient. Since the speech processor 10 is inserted into the external auditory canal, it is possible to prevent the patient who uses the cochlea implant system from feeling uncomfortable. In addition, since the microphone 12 is positioned in the external auditory canal, it is possible to minimize the exposure of the cochlea implant system and to maximally utilize the unique functions of the auricle such as the collection of sounds and the sensing of the directions of the sounds.

The transmitting unit 16 includes a modulating unit (not shown) for modulating a signal and a light emitting diode (LED) or a laser diode (LD) for emitting infrared rays in accordance with the modulated signal.

The implanted part 20 includes a receiving unit 32 for receiving an infrared signal from the speech processor 10 and for demodulating the received infrared signal, a stimulation circuit unit 34 for converting the demodulated signal into a stimulation signal, an electrode array 40 inserted into the cochlea to stimulate the auditory neurons by the stimulation signal, a coil 50 for receiving power from the outside through radio frequency (RF) power transmission, and a battery 36.

The receiving unit 32 includes a photo-detector (not shown) for detecting the infrared signal and a demodulating unit (not shown) for demodulating the detected infrared signal.

The receiving unit 32, the stimulation circuit unit 34, and the battery 36 can be provided in a hermetically sealed package 30. The package 30 is preferably formed of a biocompatible material such as metal, ceramic, sapphire glass, aluminum oxide, liquid crystal polymer (LCP), polyimide, biocompatible epoxy, silicone elastomer, or a metal alloy for medical purpose such as stainless steel and titanium alloy, and is hermetically sealed so that body fluids do not permeate into the package 30.

The package 30 of the implanted part 20 is implanted into the mastoid cavity close to the posterior wall of the external auditory canal, in order that the package 30 is implanted as close as possible to the speech processor 10. As such, the speech processor 10 and the implanted part 20 can perform the infrared data communication through the skin of the external auditory canal.

Since the speech processor 10 and the implanted part 20 perform the infrared data communication through the skin of the external auditory canal in the cochlea implant system according to the present invention, it is not necessary to use an external RF coil for transmitting the voice signal or the acoustic signal. Therefore, it is possible to prevent the patient who uses the cochlea implant system from feeling uncomfortable due to the relatively large sized coil.

Therefore, the package 30 is manufactured as small as possible. The package is preferably manufactured so that the width and the height are no more than 20 mm and that the thickness is no more than 10 mm.

An optical window 38 that can transmit the infrared light is provided in the package 30 so that the receiving unit 32 can receive the infrared light from the speech processor 10 and that the transmitting unit 33 can transfer the infrared light. The optical window can be formed of a biocompatible material and that can transmit the infrared light such as sapphire glass, Pyrex glass, biocompatible epoxy, LCP, polyimide, and silicone elastomer.

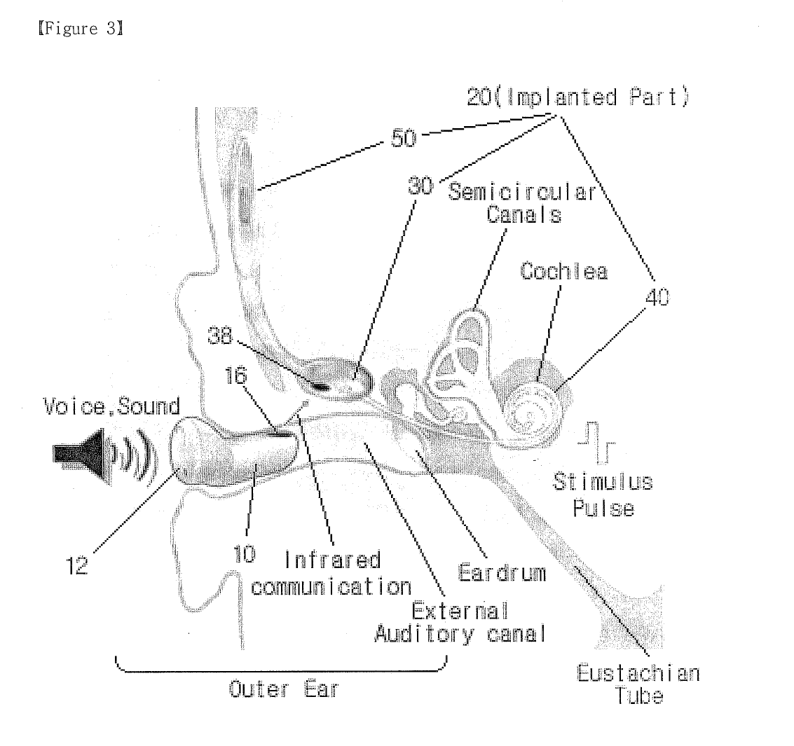

FIG. 3 is a view illustrating that the auditory neurons are stimulated by the cochlea implant system according to the present invention. Referring to FIG. 3, the microphone 12 in the speech processor 10 converts the voice signal or the acoustic signal into the electric signal and the transmitting unit 16 modulates the electric signal, converts the modulated electric signal into the infrared signal, and transfers the infrared signal to the package 30 of the implanted part 20 implanted into the inside of the body.

The receiving unit 32 of the implanted part 20 receives the infrared signal through the optical window 38 and demodulates the received infrared signal. The stimulation circuit unit 34 converts the demodulated signal into a waveform suitable for stimulating the auditory neurons.

The converted signal stimulates the auditory neurons in the cochlea implant system through the electrode array 40 inserted into the cochlea so that the patient can hear external voices or sounds.

In the cochlea implant system according to the present invention, since communications are performed between the speech processor 10 and the implanted part 20 through the infrared signal, a large amount of data can be transferred at high speed, so complicated and various speech processing methods can be applied.

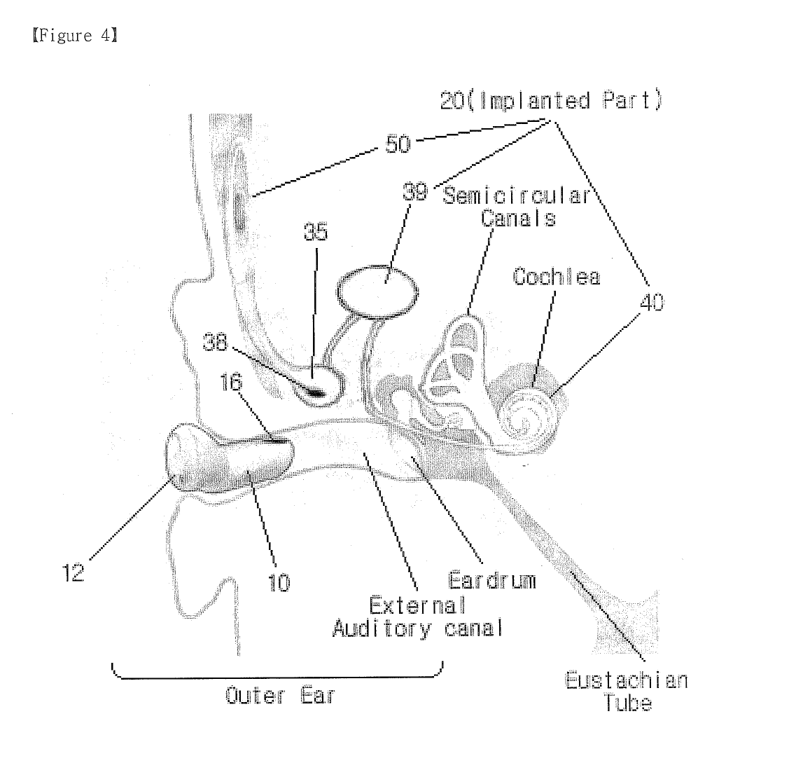

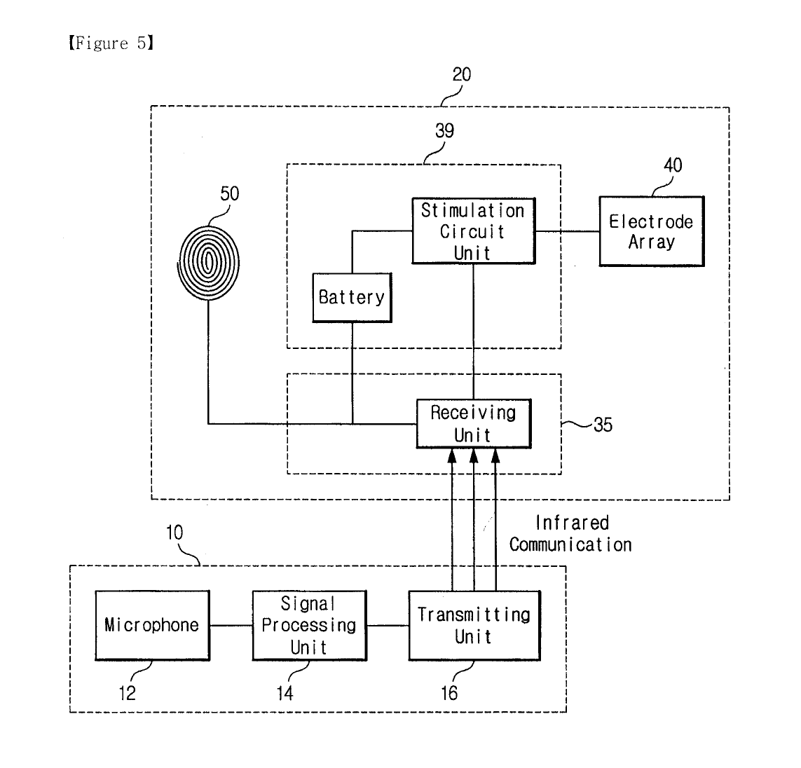

According to the above embodiment, the receiving unit 32, the stimulation circuit unit 34, and the battery are provided in one package 30. However, as an alternative, the receiving unit can be provided in separate package. FIG. 4 is a view illustrating that a cochlea implant system according to another embodiment of the present invention is implanted. FIG. 5 is a block diagram of the cochlea implant system according to another embodiment of the present invention.

In the cochlea implant system according to the present embodiment, as illustrated in FIG. 4, the infrared receiving unit 32 and the optical window 38 that transmits the infrared light are provided in a separate package 35 and the package 35 is implanted into the mastoid cavity. On the other hand, the stimulation circuit unit 34 for converting the signal received through the infrared receiving unit 32 into the stimulation signal and a battery 36 are provided in an additional package 39.

In the above structure, the sizes of the package 35 that performs the infrared data communication are minimized so that the package 35 can be implanted into a more proper position.

The coil 50 for the implanted part 20 receiving power from the outside through RF power transmission is inserted and implanted under the scalp above the temporal bone mastoid close to the mastoid cavity into which the package 30. The battery 36 can be further provided in the package 30 of the implanted part 20 and the battery receives power from the outside through the coil to be charged.

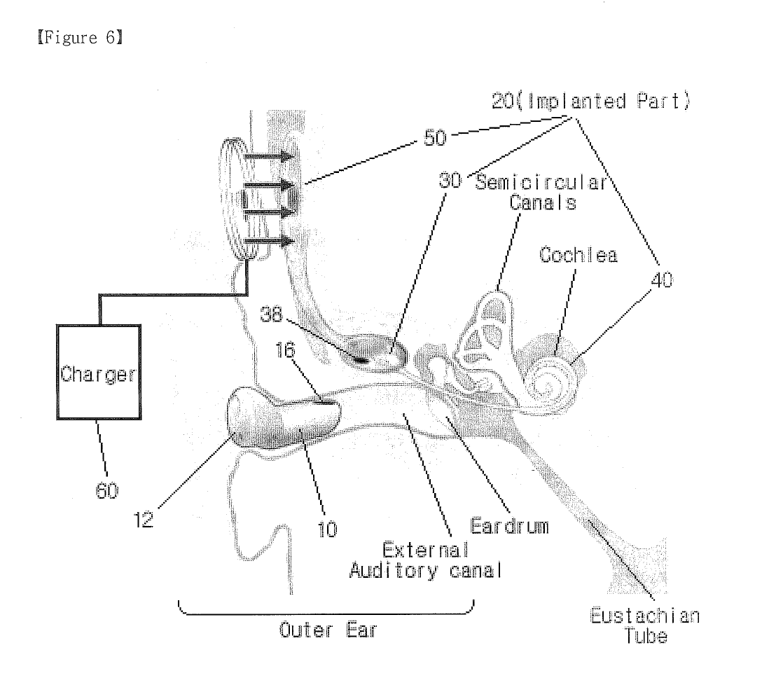

FIG. 6 is a view illustrating processes of supplying power to the implanted part 20 of the cochlea implant system according to the present invention from a charger 60 through the RF power transmission to charge the battery 36 of the implanted part 20. As illustrated in FIG. 6, the implanted part 20 can be charged through the RF power transmission between the coil 62 of the charger 60 and the coil 50 of the implanted part 20. In addition, the implanted part 20 can operate directly by receiving power through the RF power transmission between the external charger 60 including a battery (not shown) and the coil 50 of the implanted part 20.

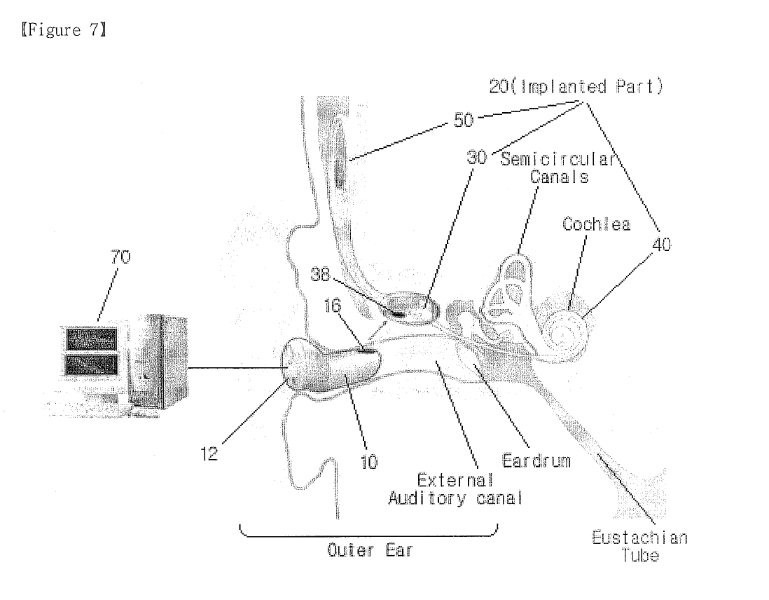

FIG. 7 is a view illustrating processes of connecting a computer 70 to the speech processor 10 to install a program in the implanted part 20 of the cochlea implant system or to upgrade the program according to the present invention. As illustrated in FIG. 7, in order to install or upgrade the program in the implanted part 20, the implanted part 20 and the speech processor 10 must perform interactive communications.

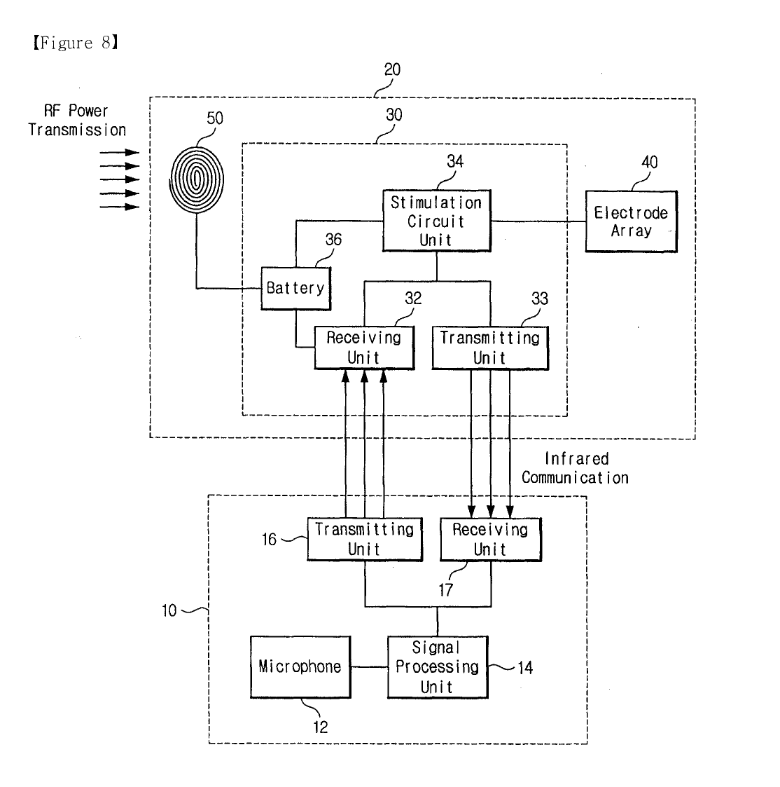

To this end, as illustrated in FIG. 8, the implanted part 20 further includes an infrared transmitting unit 33 capable of transmitting an infrared signal to the speech processor 10 and the speech processor 10 further includes an infrared receiving unit 17 capable of receiving the infrared signal from the implanted part 20. That is, each of the implanted part 20 and the speech processor 10 includes an infrared transmitting unit and an infrared receiving unit.

When the interactive communications can be performed between the implanted part 20 and the speech processor 10, information on the current status of the implanted part 20, for example electrode impedance, can be checked easily.

In particular, the implanted part 20 according to the present invention can further include a test circuit (not shown) in the package 30 for extracting information on an electrode array status and a system operation status and a measuring circuit unit (not shown) for measuring an auditory neural response induced by a stimulation. The information obtained by the test circuit and the measuring circuit unit of the implanted part 20, that is, the electrode array status information, the system operation status information, and the auditory neural response information, are modulated to a form suitable for the infrared data communication, the modulated signal is transmitted to the speech processor through the infrared transmitting unit, and the information is transmitted to the computer 70.

### INDUSTRIAL APPLICABILITY

In the cochlea implant system according to the present invention, the implanted part communicates with the speech processor through the infrared data communication and receives power from the outside through the coil to be operated or to be charged.

In particular, according to the present invention, the speech processor is inserted into the external auditory canal and the package of the implanted part is implanted into the mastoid cavity close to the posterior wall of the external auditory canal in order to perform the remote infrared data communication with the speech processor inserted into the external auditory canal.

On the other hand, the implanted part includes the coil so that the implanted part can be charged by the RF power transmission. The coil is implanted under the scalp above the temporal bone mastoid outside the external auditory canal.

Therefore, it is possible to minimize the size of the package to be implanted into the mastoid cavity of the body.

In addition, the function of a hearing aid can be combined with the cochlea implant system according to the present invention.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A cochlea implant system, comprising:
a speech processor inserted into the external auditory canal to convert a voice signal or an acoustic signal into an electric signal, to process the electric signal, and to transfer the processed electric signal to an inside of a body; and
an implanted part implanted into the mastoid cavity to receive power from an outside through radio frequency (RF) power transmission, to receive the signal from the speech processor, and to stimulate the auditory neurons in the cochlea implant system,
wherein the speech processor and the implanted part transfer and receive the signal by infrared data communication.

2. The cochlea implant system as set forth in claim 1, wherein the implanted part comprises:
a receiving unit for receiving an infrared signal from the speech processor to demodulate the infrared signal;
a stimulation circuit unit for converting the demodulated signal into a stimulation signal;
an electrode array inserted into the cochlea to stimulate the auditory neurons by the stimulation signal; and
a coil for receiving power from the outside through the RF power transmission.

3. The cochlea implant system as set forth in claim 2, wherein the receiving unit and the stimulation circuit unit are provided in a hermetically sealed package.

4. The cochlea implant system as set forth in claim 3, wherein the package is formed of metal, ceramic, sapphire glass, aluminum oxide, liquid crystal polymer (LCP), polyimide, biocompatible epoxy, silicone elastomer, stainless steel, or titanium alloy.

5. The cochlea implant system as set forth in claim 3, wherein an optical window that transmits infrared light is provided in the package.

6. The cochlea implant system as set forth in claim 5, wherein the optical window is formed of sapphire glass, Pyrex glass, biocompatible epoxy, LCP, polyimide, or silicone elastomer.

7. The cochlea implant system as set forth in claim 2,
wherein the implanted part further comprises a battery, and
wherein the battery receives power from the outside through the coil to be charged.

8. The cochlea implant system as set forth in claim 2, wherein the implanted part receives power from the outside through the coil to operate.

9. The cochlea implant system as set forth in claim 2,
wherein the receiving unit and the stimulation circuit unit are provided in separate hermetically sealed packages, and
wherein an optical window that can transmit infrared rays is provided in the package in which the receiving unit is provided.

10. The cochlea implant system as set forth in claim 1, further comprising:
a test circuit for extracting information on an electrode array status and a system operation status; and
a measuring circuit unit for measuring an auditory neural response signal induced by a test stimulus.

11. The cochlea implant system as set forth in claim 10, wherein the implanted part further comprises an infrared transmitting unit for transmitting electrode array status information, system operation status information, and auditory neural response information obtained by the test circuit and the measuring circuit unit to the speech processor in the form of an infrared signal.

12. The cochlea implant system as set forth in claim 3, wherein the package of the implanted part is manufactured so that a width and a height are no more than 20 mm and a thickness is no more than 10 mm.

13. The cochlea implant system as set forth in claim 2, wherein the coil is implanted under the scalp above the temporal bone mastoid.

14. The cochlea implant system as set forth in claim 2, wherein the receiving unit comprises a photo-detector for detecting the infrared signal and a demodulating unit for demodulating the detected infrared signal.

15. The cochlea implant system as set forth in claim 5, wherein the package and the optical window are formed of a biocompatible material.

16. The cochlea implant system as set forth in claim 1, wherein the speech processor comprises:
a microphone for converting the voice signal or the acoustic signal into an electric signal; and
a transmitting unit for modulating the electric signal, for converting the modulated electric signal into an infrared signal, and for transmitting the converted infrared signal to the inside of the body.

17. The cochlea implant system as set forth in claim 16, wherein the speech processor further comprises a receiving unit for receiving the infrared signal from the implanted part.

18. The cochlea implant system as set forth in claim 16, wherein the transmitting unit comprises:
a modulating unit for modulating a signal; and
a light emitting diode (LED) or a laser diode (LD) for emitting infrared light in accordance with the modulated signal.

19. An implanted part implanted into the mastoid cavity to receive a signal from a speech processor and to stimulate the auditory neurons in the cochlea, the implanted part comprises:
a receiving unit for receiving an infrared signal from the speech processor through the skin of the external auditory canal to demodulate the infrared signal;
a stimulation circuit unit for converting the demodulated signal into a stimulation signal;
an electrode array inserted into the cochlea to stimulate the auditory neurons by the stimulation signal; and
a coil for receiving power from the outside through radio frequency (RF) power transmission.

20. The implanted part as set forth in claim 19, further comprising:
a test circuit for extracting information on an electrode array status and a system operation status;
a measuring circuit unit for measuring an auditory neural response signal induced by a stimulus; and
an infrared transmitting unit for transmitting electrode array status information, system operation status information, and auditory neural response information obtained by the test circuit and the measuring circuit unit to the speech processor in the form of the infrared signal.

21. A method of stimulating the auditory neurons, comprising:
a speech processor converting a voice signal or an acoustic signal into an electric signal;
modulating the converted electric signal into an infrared signal;
transmitting the infrared signal to an implanted part implanted into the inside of a body through the skin of the external auditory canal;
the implanted part receiving the transferred infrared signal;
demodulating the received infrared signal;
converting the demodulated signal into a waveform suitable for stimulating the auditory neurons;
stimulating the auditory neurons by the converted signal; and
supplying power to the implanted part through RF power transmission.

22. The method as set forth in claim 21, further comprising:
the implanted part measuring a stimulus waveform induced in an electrode and a system internal operation signal and modulating the measured stimulus waveform and system internal operation signal;
recording auditory neural response induced by an electric stimulus and modulating the recorded auditory nerve response; and
transmitting the modulated signal as the infrared signal to the outside.
